SEQUENCE LISTING (8 sequences)

SEQ ID NO: 1 - DNA, 25 bp, Artificial Sequence
  Primer
cgacgacttctcccgccgctaccgc

SEQ ID NO: 2 - DNA, 25 bp, Artificial Sequence
  Primer
ccgcatgctggggccgtacagttcc

SEQ ID NO: 3 - DNA, 20 bp, Artificial Sequence
  Primer
gtgcaccaaggtgccggaac

SEQ ID NO: 4 - DNA, 20 bp, Artificial Sequence
  Primer
tcagcccatcttcttccaga

SEQ ID NO: 5 - DNA, 20 bp, Artificial Sequence
  Primer
agtgctcgcagctcatacct

SEQ ID NO: 6 - DNA, 20 bp, Artificial Sequence
  Primer
gagtccattgattcgcttcc

SEQ ID NO: 7 - DNA, 20 bp, Artificial Sequence
  Primer
gtggggcgccccaggcacca

SEQ ID NO: 8 - DNA, 24 bp, Artificial Sequence
  Primer
ctccttaatgtcacgcacgatttc

(19) United States
(12) Patent Application Publication
(10) Pub. No.: US 2011/0071094 A1
(43) Pub. Date: Mar. 24, 2011

(54) (D)-ALLOSE INDUCING PROGRAMMED CELL DEATH IN HORMONE REFRACTORY PROSTATE CANCER LINES

(75) Inventors: MYEONG OK KIM, JINJU-SI (KR); BONG CHUL CHUNG, SEOUL (KR)

(73) Assignee: INDUSTRY-ACADEMIC COOPERATION FOUDATION, GYEONGSANG NATIONAL UNIVERSITY, JINJU-SI (KR)

(21) Appl. No.: 12/911,279

(22) Filed: Oct. 25, 2010

Related U.S. Application Data
(63) Continuation of application No. PCT/KR2008/007320, filed on Dec. 10, 2008.

(30) Foreign Application Priority Data
Apr. 24, 2008 (KR) 10-2008-0038010

(51) Int. Cl. A61K 31/7004; C07H 3/02; C12N 5/09; A61P 35/00

(52) U.S. Cl. 514/23; 536/1.11; 435/375

(57) ABSTRACT

The present disclosure relates allose inducing programmed cell death in hormone refractory prostate cancer cell lines. More particularly, the present disclosure relates to a D-allose inducing programmed cell death in hormone refractory prostate cancer cell line of DU145 in dose-dependent manner by repressing cell growth and inhibiting cell cycle as well as by reducing expression of caspase-3 and poly (ADP-ribose) polymerase (PARP).

### CROSS-REFERENCE TO RELATED APPLICATIONS

This application is a continuation application under 35 U.S.C. §365(c) of International Application No. PCT/KR2008/007320, filed Dec. 10, 1008, designating the United States. This application further claims for the benefit of the earlier filing dates under 35 U.S.C. §365(b) of Korean Patent Application No. 10-2008-0038010 filed Apr. 24, 2008. This application incorporates herein by reference the International Application No. PCT/KR2008/007320 and the Korean Patent Application No. 10-2008-0038010 in their entirety.

### BACKGROUND

1. Technical Field

The present disclosure relates to allose inducing programmed cell death in hormone refractory prostate cancer cell lines.

2. Discussion of Related Technology

Because of high prevalence, mortality, and unsatisfactory treatment options available at this time, hormone-refractory prostate cancer (HRPC) becomes the second leading cause of cancer related death in men in the United States. In man, prostate homeostasis is dependent on the equilibrium between cell proliferation through cell division, and cell loss through apoptosis (programmed cell death, PCD). Defects in apoptosis signaling may hinder this balance, thereby enhancing tumor development and metastasis to distant sites like seminal vesicle, urinary bladder, rectum, bone, lymph node via blood and brain in a more aggressive form resulting in lethality. The metastatic prostate cancer (PC) is initially responsive to standard treatment regimes such as hormone therapy, radiotherapy, chemotherapy, and prostectomy. However, in late stage, this metastatic disease is shifted from androgen dependence to androgen independence, which is often provoked by androgen-ablation therapy.

The bcl-2 family of proteins (both anti- and pro-apoptotic members) is of special importance in this regards, as they can heterodimerize and seemingly titrate one another's function, thus controlling programmed cell death signaling pathway via their relative concentrations. These proteins function at a common part of the apoptosis signaling pathway by acting as a checkpoint upstream of caspases, executors of apoptosis, and mitochondrial dysfunction. The apoptosis signaling pathway also considers mitochondrial permeability transition due to loss of mitochondrial transmembrane potential (Δψm), and the release of cytochrome C (cyt C) as the central events initiating the execution of programmed cell death.

Mitochondrial depolarization in apoptosis is thought to be associated with the permeability transition (PT); an event involving the formation of a voltage-dependent anion channel (VDAC) in the mitochondrial membranes, which is also blocked by Bcl-2. The calcium concentration in cytosol ([Ca²⁺]c) is the prerequisite for post mitochondrial events involved in downstream caspase activation and subsequent induction of apoptosis in human prostate cancer cell line. The expression of anti-apoptotic bcl-2 is often enhanced in late stage HRPC that promotes cell survival by preventing interaction of Apaf-1-cyt C complexes, result in caspases (caspase 9 to caspase 3) inactivation. Bcl-2 over expression due to recurrence of androgen independence further produces chemoresistant phenotype by genetic alterations. Therefore, new approaches for the management of HRPC are urgently needed.

Rare sugars are defined by the International Society of Rare Sugars (ISRS) as monosaccharides that are rarely distributed in nature (The 1st International Symposium of ISRS, Takamatsu, Japan, 2002). Because of scarcity and cost, the biological function of rare sugars has not been investigated in detail, except their usages as low-calorie carbohydrate sweeteners and bulking agents. However, after “Izumoring”, enormous research with rare sugars especially D-allose (C-3 epimer of glucose) has become possible. D-Allose inhibited segmented neutrophil production and lowered platelet count without any side effects, and may play a pivotal role in organ or tissue transplantation as an immunosuppressant. The novel inhibitory effects of D-allose on the production of reactive oxygen species (ROS) from neutrophils and on the proliferation of various cancer cell lines were also reported. But, till today there is no report on the effect of D-allose on prostate cancer treatment, especially late stage HRPC. The foregoing discussion is only to provide background information of the invention disclosed herein and does not constitute an admission of prior art.

### SUMMARY

One aspect of the present invention is related to a composition for inducing apoptosis in prostate cancer cells, the composition comprising D-allose. In some embodiments, the prostate cancer cells may comprise human hormone refractory prostate cancer cells. In some other embodiments, D-allose may induce apoptosis in prostate cancer cells by i) lowering a mitochondrial transmembrane potential (Δψm); ii) elevating a calcium concentration in cytosol ([Ca²⁺]c); iii) cleaving caspase 3 and poly (ADP-ribose) polymerase (PARP); and iv) releasing cytochrome C (cyt C). In still some other embodiments, D-allose may induce apoptosis by modulating expression at a transcription level of apoptosis-related genes through increasing expression of Bax and decreasing expression of Bcl-2.

Another aspect of the present invention is related to a method of inducing apoptosis in prostate cancer cells, comprising contacting the composition to prostate cancer cells. In some embodiments, the composition may be applied at 20˜40 mM per 1×10⁵ cells of prostate cancer cells.

Still another aspect of the present invention is related to a pharmaceutical composition for prevention or treatment of prostate cancer by inducing apoptosis and inhibiting cell growth of prostate cancer cells, the composition comprising D-allose and a pharmaceutically acceptable carrier. In some embodiments, the prostate cancer may be hormone refractory prostate cancer.

Still another aspect of the present invention is related to a composition for health functional food by inducing apoptosis in prostate cancer cells to prevent and improve prostate cancer, the composition comprising food, D-allose and a sitologically acceptable food additive.

Still another aspect of the present invention is related to a method of treating prostate cancer comprising administering to a person in need of such treatment D-allose in an amount sufficient to induce apoptosis and inhibit cell growth of prostate cancer cells. In some embodiments, the prostate cancer may be hormone refractory prostate cancer.

### DESCRIPTION OF DRAWINGS

FIG. 1 shows effects of D-allose on cell growth, apoptosis and cell cycle distribution of human HRPC cell line, DU145. Cells are exposed to 0, 1, 5, 10, 20 and 40 mM of D-allose for up to 72 h and then assessed for cell viability (by MTT assay), programmed cell death (by DAPI/Annexin-V-FLUOS staining) and cell cycle distribution (by PI staining). Detail procedures are mentioned in Examples. (A) Growth inhibitory effects are expressed as percentage of cell growth inhibition induced by D-allose for 24, 48 and 72 h relative to the untreated controls. Results represent the mean±SEM of the three independent experiments. *P<0.05, **P<0.01, ^{†}P<0.001. (B) Cells are in the culture media with and without D-allose treatments for 48 h×400. (C) Net apoptosis induction by D-allose for 24, 48 and 72 h relative to the untreated controls. Results represent the mean±SEM of the three independent experiments. *P<0.05, **P<0.01, ^{†P<}0.001. (D) Apoptotic cells by DAPI staining with and without D-allose treatments for 48 h×400. The confocal images are the basis of calculation of apoptotic indices as mentioned in (C). (E) Flow cytometric analysis of apoptotic cells by annexin-V/PI staining upon treatments with D-allose for 48 and 72 h. Results are the true representative of the triplicate experiments. (F) Distribution of D-allose treated cells for 48 h in the different phases of cell cycle. Percentage of cells in the G0-G1, G2-M and S phases are calculated with ModFit LT software, version 2.0 (Beckon Dickinson), and are presented in the right side of the histograms. Results are the true representative of the triplicate experiments.

FIG. 2 shows effects of D-allose on Bcl-2, Bax, caspase 3 and PARP expression levels in human HRPC cell lines, DU145. Cells are exposed to 0, 20 and 40 mM of Dallose for 72 h. Detail procedures are mentioned in Examples. (A) After exposure cells are lysed and assayed for Bcl-2, Bax, full length caspase 3 and cleaved caspase 3, full length PARP and cleaved PARP proteins expression by Western blot analysis. β-actin expression indicates uniform loading in each lane. Results are the true representative of the triplicate experiments. (B) Densitometric evaluations of Bcl-2, Bax, full length caspase 3 and cleaved caspase 3, full length PARP and cleaved PARP protein bands of (A), respectively by Sigma Gel, version 1.0. Results represent the mean±SEM of the three independent experiments. *P<0.05, **P<0.01, ^{†}P<0.001. (C) Cells with and without D-allose treatments are subjected to analysis of Bcl-2, Bax and caspase 3 mRNAs by RT-PCR, where β-actin is taken as loading control. Results are the true representative of the triplicate experiments. (D) Densitometric analysis of Bcl-2, Bax and caspase 3 mRNA bands of (C), respectively by Molecular Analyst™, version 1.4.1 (Bio-Rad). Results represent the mean±SEM of the three independent experiments. *P<0.05, **P<0.01, ^{†P<}0.001.

FIG. 3 shows effects of D-allose on mitochondrial Δψm, cyt C expression and nuclear morphology in human HRPC cell lines, DU145. Cells are exposed to 0, 20 and 40 mM of D-allose for up to 72 h. Detail procedures are mentioned in Examples. (A) The immunofluorescence of DU145 cell line doublestained for cyt C (FITC-labeled cyt C antibody, green) and chromatin (PI, red) upon treatments with D-allose for 48 h. Yellow color (green+red; merge image) indicates release of cyt C from mitochondria into cytosol and chromatin condensation in the same cells, as detected by confocal microscopy×600 (0 mM), ×400 (20 mM), ×3500 (40 mM). DU145 cell line treated with FITC-labeled secondary antibody alone is considered as negative control×600. (B) The translocation of cyt C is observed in the confocal image of double-stained DU145 cell line after treatments with 40 mM of D-allose for 72 h. Mitochondria are probed with FITC-labeled cyt C antibody (green) and nucleus is counterstained with PI (red) ×3000. The fluorescence micrographs of mitochondria (JC-1, green) in DU145 cell line with and without D-allose treatments for 72 h×600 (0 mM), ×450 (20 and 40 mM). (C) Flow cytometric analysis of mitochondrial Δψm by JC-1 staining upon treatments with D-allose for 48 and 72 h. Loss of mitochondrial Δψm is associated with increase in FL1 fluorescence. Results are the true representative of the triplicate experiments. (D) Quantification of cells with low mitochondrial Δψm (as the percentage of total cell population) induced by D-allose for 24, 48 and 72 h, as detected by flow cytometry. Results represent the mean±SEM of the three independent experiments. *P<0.05, **P<0.01, ^{†}P<0.001.

FIG. 4 shows effects of D-allose on [Ca²⁺]c in human HRPC cell lines, DU145. Cells are exposed to 0, 20 and 40 mM of D-allose for 48 h followed by fura-2 AM labeling and fluorescence spectra for [Ca²⁺]c are measured by luminescence spectrophotometer. Detail procedures are mentioned in Examples. Inset: The fluorescence micrographs of fura-2 AM labeled single DU145 cell with (A, 20 mM) and without (B, 0 mM) D-allose treatments for 48 h×2500.

FIG. 5 shows proposed mechanism of D-allose as an antiproliferative agent mediates through induction of programmed cell death in late stage human HRPC cell line, DU145. D-allose in vitro exposure to human HRPC cell lines could alter Bcl-2/Bax ratio that decreases mitochondrial Δψm and promotes cyt C release from mitochondria leading to activation of downstream caspase 3 and finally cleavage of the caspase 3-target protein PARP, result in chromatin condensation, a hallmark of programmed cell death. High [Ca²⁺]c is the earliest biochemical event of this mitochondria mediated intrinsic pathway of apoptosis. On the other hand, Bcl-2 over expression prevents leakage of Ca²⁺ from its cellular ER store, lowers [Ca²⁺]c, subsequently stops cyt C release from mitochondria, and ultimately blocks apoptosis (programmed cell death). However, it would be interesting to investigate in future whether D-allose has any role in the very upstream events of intrinsic pathway of apoptosis in HRPC cell lines involving DU145.

### DETAILED DESCRIPTION OF INVENTIVE FEATURES AND EMBODIMENTS

Therefore, the present inventors have made an effort on investigating the dose and time-dependent effects of D-allose on the proliferation of late stage human HRPC using DU145 cell line in vitro, which are highly tumorigenic and chemotherapy-resistant. The present inventors confirmed the mechanism of D-allose induced apoptosis in DU145 cell line by modulation of pro- and anti-apoptotic Bcl-2 family members, Bax and Bcl-2, in favor of execution of programmed cell death, which is accompanied by mitochondrial cyt C release along with concomitant alteration of mitochondrial Δψm, elevation of [Ca²⁺]c, and cleavage of caspase-3 and poly (ADP-ribose) polymerase (PARP).

Various features and embodiments of the present invention relate to estimating dosage and time-dependent effects of D-allose on the proliferation of late stage human HRPC using DU145 cell line in vitro, which are highly tumorigenic and chemotherapy-resistant. Also, the present invention relates to estimate the mechanism of D-allose induced apoptosis in DU145 cell line by modulation of pro- and anti-apoptotic Bcl-2 family members, Bax and Bcl-2, in favor of execution of programmed cell death, which is accompanied by mitochondrial cyt C release along with concomitant alteration of mitochondrial Δψm, elevation of [Ca²⁺]c, and cleavage of caspase-3 and poly (ADP-ribose) polymerase (PARP).

It is an effect of some embodiments of the present invention to provide the dose and time-dependent effects of D-allose on the proliferation of late stage human HRPC using DU145 cell line in vitro, which are highly tumorigenic and chemotherapy-resistant. Also, it is an effect of some other embodiments of the present invention to provide the mechanism of D-allose induced apoptosis in DU145 cell line by modulation of pro- and anti-apoptotic Bcl-2 family members, Bax and Bcl-2, in favor of execution of programmed cell death, which is accompanied by mitochondrial cyt C release along with concomitant alteration of mitochondrial Δψm, elevation of [Ca²⁺]c, and cleavage of caspase-3 and poly (ADP-ribose) polymerase (PARP).

One aspect of the present invention is to provide D-allose for the induction of apoptosis and inhibition of proliferation in hormone refractory prostate cancer cell lines of DU145 in vitro.

Further, said D-allose induces apoptosis in hormone refractory prostate cancer cell lines by i) lowering the mitochondrial transmembrane potential (Δψm); ii) releasing the cytochrome C (cyt C); iii) cleaving the caspase 3 and poly (ADP-ribose) polymerase (PARP); and iv) elevating the calcium concentration in cytosol ([Ca²⁺]c).

Further, said D-allose induces apoptosis in hormone refractory prostate cancer cell lines by modulating expression at the transcription level of apoptosis-related genes through increasing expression of pro-apoptotic family, Bax and decreasing expression of anti-apoptotic family, Bcl-2.

Further, another aspect of the present invention is to provide an effective dose of D-allose is 20˜40 mM per 1×10⁵ cells of DU145 cell line.

Development of effective agents for treatment of hormone-refractory prostate cancer (HRPC) has become a national medical priority. D-Allose is a monosaccharide (C-3 epimer of glucose) distributed rarely in nature; because of its scarcity and cost, the biological effect has hardly been studied. In some embodiments of the present invention, we demonstrated the inhibitory action of D-allose on proliferation of human HRPC cell lines, DU145 in a dose- and time-dependent manner.

In vitro treatment of D-allose resulted in the alteration of Bcl-2/Bax ratio in favor of apoptosis (programmed cell death, PCD) in the DU145 cell line, which was associated with the lowering of mitochondrial transmembrane potential (Δψm) and the release of cytochrome C (cyt C), the cleavage of caspase 3 and poly (ADP-ribose) polymerase (PARP), and the elevation of calcium concentration in cytosol ([Ca²⁺]c).

D-Allose also induced G1 phase arrest of the cell cycle in DU145 cell line. This invention for the first time suggested the antiproliferative effect of D-allose through induction of programmed cell death in HRPC cell line, DU145 which could be due to the modulation of mitochondria mediated intrinsic apoptotic pathway.

Hereinafter, some embodiments of the present invention are explained in detail.

D-Allose Inhibited Cell Growth in DU145 Cell Line

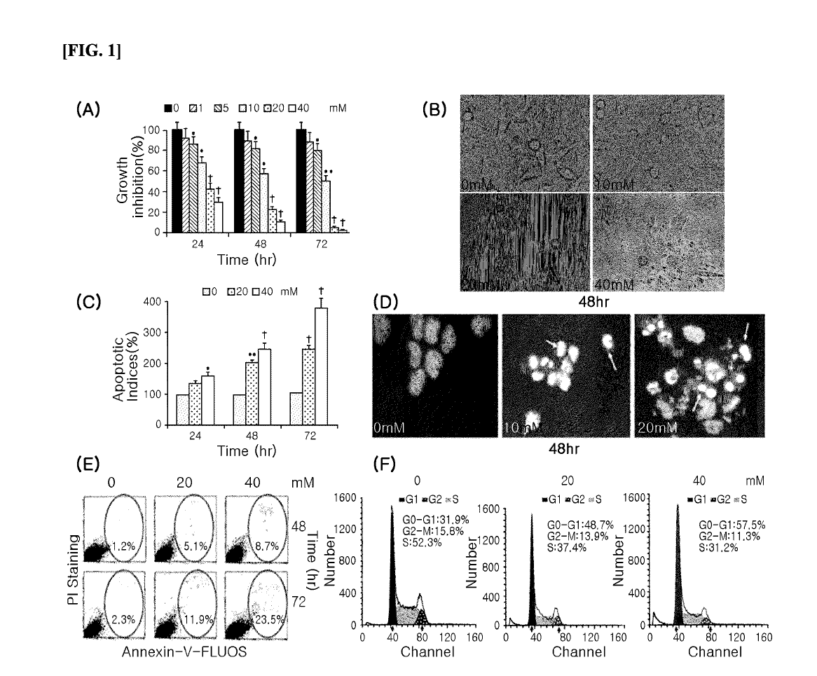

One aspect of the present invention is to investigate whether rare sugar D-allose imparts anti-proliferative effects against human HRPC cell line. Therefore, DU145 cell line was treated with different concentrations of D-allose and cell growth was assayed by MTT assay. D-Allose treatment (0-40 mM) resulted in a significant dose-dependent inhibition of cell growth in DU145 (FIGS. 1A, B) cell line as compared to the control. D-Allose treatment also resulted in time-dependent inhibition of DU145 cell growth, which was more pronounced at 72 h post-treatment in contrast with the control (FIG. 1A). We also examined the effect of D-allose on the growth of human NPE cell line, PrEC.

Our results revealed PrEC cell line showed no remarkable effect. We further studied the effect of D-glucose on proliferation of the DU145 cell line, which revealed significant increase of cell growth in a dose-dependent manner.

D-Allose Induced Programmed Cell Death in DU145 Cell Line

In order to determine whether D-allose-induced growth inhibition is due to programmed cell death, we evaluated the apoptotic indices at the indicated times of treatment (i.e. 24, 48 and 72 h) by microscopical analysis. For this purpose DNA intercalating dye DAPI was used on human HRPC cell line, DU145 and NPE cell line, PrEC. As expected, very negligible effect was observed in PrEC cell line in contrast with the other cancer cell line studied (FIGS. 1C, D). D-Allose induced significant dose- and time-dependent programmed cell death in DU145 (FIG. 1C) cell line, as revealed by DAPI staining.

The extent of apoptosis was quantified by flow cytometric analysis where DU145 cell line was labeled with annexin-V and PI. In DU145 cell line, D-allose of 20 and 40 mM caused 5.1% and 8.7% of apoptotic cells in 48 h treated groups, and 11.9% and 23.5% of apoptotic cells in 72 h treated groups, as compared to the respective untreated controls i.e. 1.2% and 2.3% of apoptotic cells in 48 and 72 h control groups, respectively (FIG. 1E). Therefore, induction of apoptosis (programmed cell death) was significantly higher when high dose of D-allose (40 mM) was applied for 72 h. Our invention also revealed no remarkable effect of D-glucose on the programmed cell death in DU145 cell line.

D-Allose Induced G1 Phase Arrest of Cell Cycle in DU145 Cell Line

To assess whether D-allose induced growth inhibition and apoptosis is mediated through the alterations in cell cycle, the cell cycle analysis of DU145 cell line was performed after treatments with various concentrations of D-allose for 48 and 72 h. Compared with the untreated control (31.9% cells in G0-G1 phase), D-allose treatment for 48 h showed significant arrest of cells in G0-G1 phase of the cell cycle (48.7% cells at 20 mM concentration that further increased to 57.5% at 40 mM concentration) (FIG. 1F). Further, the remarkable increase in G0-G1 cell population was accompanied with a concomitant decrease of cell number in G2-M phase and S phase of the cell cycle (FIG. 1F). D-Allose treatment for 72 h also maintained the similar trend (data not shown).

D-Allose Altered Bax and Bcl-2 Protein Expression in DU145 Cell Line

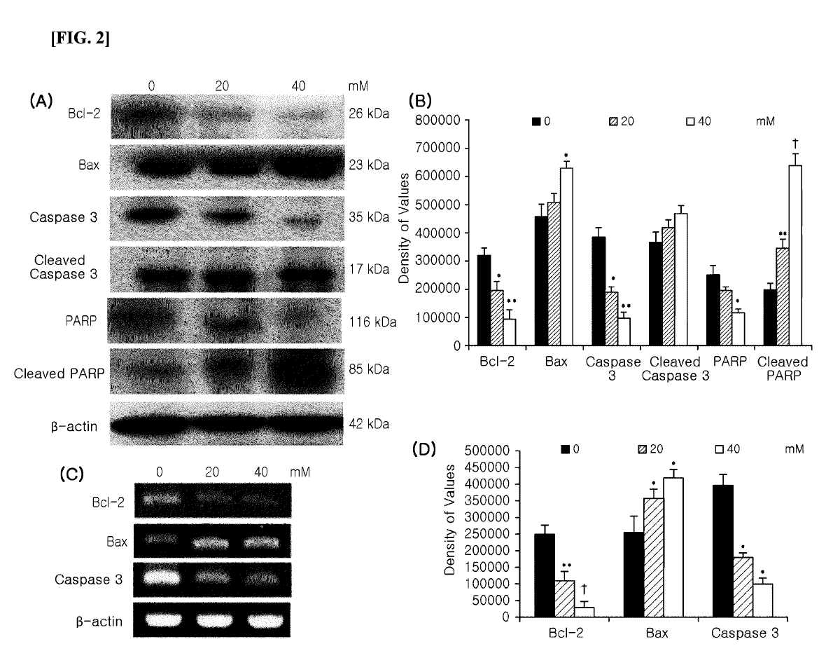

D-Allose altered Bax and Bcl-2 proteins expression in DU145 cell line Pro-apoptotic Bax and anti-apoptotic Bcl-2 are known to play a crucial role in apoptosis; therefore, the dose-dependent effect of D-allose on protein levels of Bax and Bcl-2 in DU145 cell line was studied. The Bcl-2 protein expression was significantly decreased by D-allose treatment at 20 and 40 mM concentrations for 72 h, while Bax protein expression was remarkably increased at 40 mM concentration of D-allose under the same condition in DU145 (FIGS. 2A, B) cell line. Overall, the treatment of cells with D-allose resulted in a remarkable dose-dependent shift (i.e. 0.896 at 0 Mm, 0.335 at 20 Mm, 0.126 at 40 Mm) in Bcl-2/Bax ratio, suggesting induction of programmed cell death in human HRPC cell line, DU145.

D-Allose Altered Bax and Bcl-2 mRNAs Expression in DU145 Cell Line

To demonstrate that up-regulation of Bax and Bcl-2 proteins in DU145 cell line by D-allose correlated with an alteration in the amount of Bax and Bcl-2 mRNAs, RT-PCR analysis was performed. As shown in FIGS. 2C and D, the treatment D-allose in DU145 cell line for 72 h significantly reduced Bcl-2 mRNA expression in a dose-dependent manner, whereas Bax mRNA expression showed the opposite effect. Also, the expression pattern of both the pro- and anti-apoptotic mRNAs in DU145 cell line exhibited the similar trend with that of the corresponding protein levels, confirming the role of D-allose during induction of programmed cell death in the human HRPC cell line at the transcript level of programmed cell death-related genes, Bax and Bcl-2.

D-Allose Induced Cyt C Release and Alteration of Mitochondrial Δψₘ With Subsequent Cleavage of Caspase 3 and PARP

The process of programmed cell death may involve disruption of mitochondrial function through the release of cyt C from mitochondria, which interacts with Apaf-1 leading to activation of caspases and subsequent cleavage of PARP; therefore, the dose-dependent effect of D-allose on caspase 3 and PARP protein levels in DU145 cell line were studied. As shown in FIGS. 2A and B, the different concentrations of D-allose (0, 20 and 40 mM) for 72 h resulted in a remarkable dose-dependent decrease in the full length caspase 3 and PARP proteins expression with concomitant increase of their cleaved products in DU145 cell line. Also, D-Allose treatment for 72 h further revealed the similar type of dose-dependent effect on caspase 3 mRNA expression in DU145 cell line (FIGS. 2C, D), as compared to the corresponding protein level (FIGS. 2A, B).

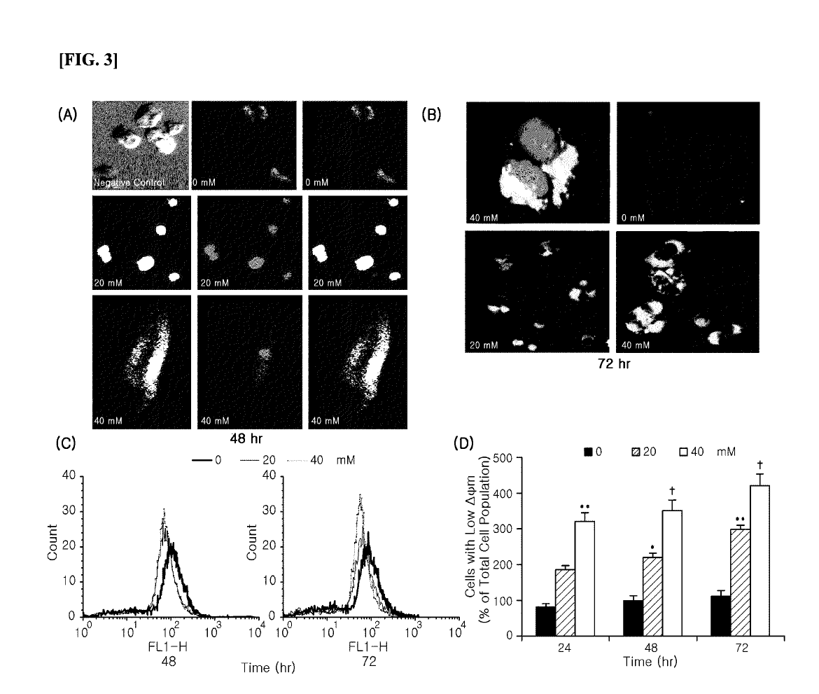

The dose- and time-dependent effects of D-allose on the release of cyt C from mitochondria and condensed chromatin in DU145 cell line were well established in some embodiments of the present invention by immunofluorescence technique (FIGS. 3A, B). The confocal laser microscopy revealed a diffuse staining pattern of cyt C, suggesting its release from mitochondria (cyt C, green) along with chromatin condensation (PI, red) upon treatments with different concentrations of D-allose for 48 h (FIG. 3A). The merge image (red+green=yellow) indicated the release of cyt C from mitochondria into cytosol and chromatin condensation as a result of D-allose treatment in the same DU145 cell line (FIG. 3A). Further, the translocation of cyt C was observed in the confocal image of double-stained DU145 cell line after treatments with 40 mM of D-allose for 72 h, where mitochondria were probed with an FITC-labeled cyt C antibody (green) and nucleus was counterstained with PI (red) (FIG. 3B).

Since Bcl-2 is located on the mitochondrial membrane and involved in the regulation of PT and the cyt C release, the present inventors also examined the mitochondrial Δψm in D-allose treated DU145 cell line for 24, 48 and 72 h by use of fluorescent dye JC-1. Uptake of JC-1 into mitochondria was monitored by flow cytometry, where loss of mitochondrial Δψm was associated with increase in FL1 fluorescence (FIG. 3C). As shown in FIGS. 3C and D, treatment of DU145 cell line with D-allose resulted in the appearance of a remarkable subpopulation of cells with low mitochondrial Δψm in a dose- and time-dependent manner. Further, the fluorescence micrographs of JC-1 stained mitochondria (green) in DU145 cell line with and without D-allose treatments for 72 h were supported by the flow cytometrical data shown in FIGS. 3C and D.

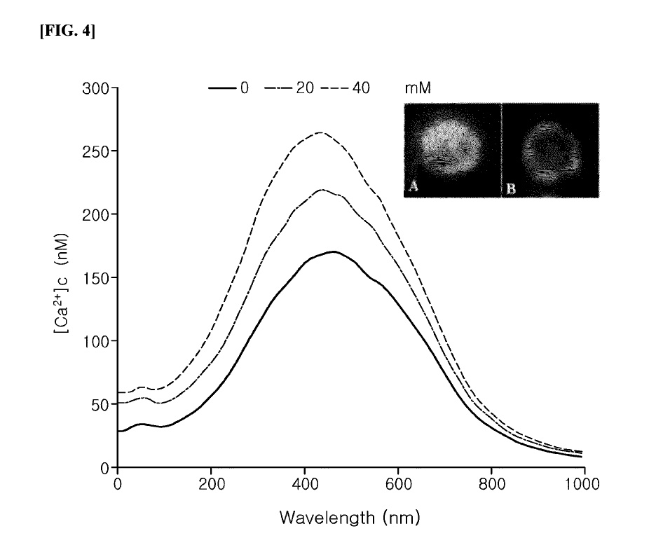

D-Allose Increased [Ca²⁺]_{c} in DU145 Cell Line

[Ca²⁺]c is the prerequisite for post-mitochondrial events including caspase activation and subsequent programmed cell death in human prostate cancer cell line. The dose-dependent effect of D-allose on [Ca²⁺]c for 48 and 72 h was considered in some embodiments of the present invention. As shown in FIG. 4A, [Ca²⁺]c was remarkably elevated in DU145 cell line at 20 and 40 mM concentrations of D-allose for 48 h. These results were further confirmed by the fluorescence micrographs of fura-2 labeled single DU145 cell. D-Allose treatment for 72 h also maintained the similar trend (data not shown). Overall, the dose- and time-dependent effect of D-allose suggesting the induction of programmed cell death in human HRPC cell line, DU145 that was triggered by the increase of [Ca²⁺]c.

The search for promising agents that could reduce the incidence and burden of cancer has become increasingly important in recent years. Immense interest has been generated for rare sugars especially D-allose in view of the role in attenuating the risk of developing cancer. The role of D-allose on HRPC needs to be addressed as PC responds initially to androgen ablation and later converts to hormone-resistant state. The recurrence of androgen independence often leads to alterations of several programmed cell death -related genes; results in anti apoptosis and chemoresistance. In this regard, DU145 cells are unique in vitro models of late stage HRPC that are highly resistant to chemotherapeutic agents. By using DU145 cell line, the present inventors showed the potentiality of D-allose to inhibit cellular growth in dose- and time-dependent manner. In contrast, D-glucose remarkably increases cell growth without any prominent effect on the programmed cell death in the human HRPC cell line, DU145. It is already known that some monosaccharides govern many cellular functions including strong inhibition on cell proliferation either in vivo or in vitro conditions because of their sugar structure. Since D-allose is the C-3 epimer of D-glucose, therefore, in the light of above observations it can be said that the novel anti-proliferative effect of D-allose on human HRPC cell lines might be associated with its sugar structure.

Whether D-allose-induced growth inhibition is due to programmed cell death, the present inventors next evaluated apoptosis in DU145 cell line. Apoptosis is a physiological process by which cells are removed when as agent damages their DNA. Apoptosis represents a discrete manner of programmed cell death that differs from necrosis and is regarded as an efficient way to eliminate damaged cells. Agents that can modulate apoptosis may be able to affect steady-state cell population, which may be useful in cancer therapy. The data in some embodiments of the present invention demonstrated that D-allose induces significant dose- and time-dependent programmed cell death in DU145 cell line, which was remarkably higher when high dose of D-allose was applied for 72 h. This observation verified by chromatin condensation, Bcl-2/Bax ratio, caspase 3 and PARP cleavage, fluorescence microscopy, and flow cytometry. This is an important finding in some embodiments of the present invention since modulation of apoptotic response finally suggests a novel mechanism based therapeutic approach towards late stage HRPC.

As cell cycle regulation and apoptosis are closely associated phenomena, in next series of experiment the present inventors analyzed the cell cycle phase distribution of DU145 cell line by flow cytometry. Compared with the untreated controls, D-allose treatment resulted in remarkable dose- and time-dependent increase of cell numbers in G0-G1 phase with a concomitant decrease of cell population in G2-M and S phase of the cell cycle, suggesting D-allose induce antiproliferative effect and programmed cell death in DU145 cell line involves cell cycle arrest at G1 phase.

Since sequential expression and activation of cyclins, cdks and cdk inhibitors play a crucial role in the regulation of eukaryotic cell cycle, therefore involvement of cyclin-cdk machinery during induction of cell cycle arrest and programmed cell death by D-allose needs to be studied further.

The Bcl-2 family members, pro-apoptotic Bax and anti-apoptotic Bcl-2 are the critical regulators of apoptotic pathway. Bcl-2 has been found in very high levels in many human tumors and carcinoma cells, over expression of which results in resistance to apoptosis that may lead to chemoresistance. Bcl-2 forms a heterodimer with Bax and neutralizing its effects. Also, the PC marker, Bcl-2/Bax ratio dictates a cell's susceptibility to undergo apoptosis under various experimental conditions. In some embodiments of the present invention, a decrease in Bcl-2 protein with consequent increase in Bax protein expression in DU145 cell line after D-allose treatment caused a significant dose-dependent shift in Bcl-2/Bax ratio towards induction of programmed cell death. Further, in some other embodiments of the present invention the expression of Bax and Bcl-2 mRNAs maintain the similar trend with that of their corresponding proteins, suggesting up regulation of Bax and Bcl-2 proteins occur at the transcript level during induction of programmed cell death in DU145 cell line by D-allose. However, the result obtained in this invention that D-allose induce apoptosis is mediated through Bcl-2 family of proteins, Bax and Bcl-2, is in contrast with the previous studies, which may be due to the differences in experimental design and cancer cell line used.

Previous studies showed that alteration in the Bcl-2/Bax ratio promotes cyt C release from mitochondria, which activates Apaf-1, allowing it to bind to and activate caspase 9. Caspase 9 then cleaves and activates downstream caspase 3, resulting in apoptosis. Alternatively, it is possible that the lack of caspase 9 activation could result from the failure to interact with Apaf-1-cyt C complexes, as influenced by Bcl-2 over expression. On the other hand, due to its unique location on mitochondrial membrane, Bcl-2 stabilizes the mitochondrial Δψm and inhibits cyt C translocation from the intermembrane space to cytosol, result in blockage of downstream caspase 3 activation and subsequent programmed cell death process.

In some embodiments of the present invention, the dose-dependent shift in Bcl-2/Bax ratio towards induction of programmed cell death in DU145 cell line after D-allose treatments is associated with mitochondrial cyt C release and a decrease in Δψm, suggesting opening of the mitochondrial PT pore. These events are linked to the VDAC, a component of the PT pore, and its interaction with Bax. Since Bcl-2 functionally inactivate Bax by heterodimerization, decrease expression of Bcl-2 in DU145 cell line by D-allose treatment lowers the threshold at which the execution of apoptosis occurs.

This whole event is further associated with the appearance of condensed chromatin in D-allose treated DU145 and cell line, a hallmark of apoptosis usually succeeding mitochondrial events. Activation of caspase 3-like protease, which is generally involved in the degradation of death substrates like PARP and constitutes the execution phase of programmed cell death controlled by Bcl-2, is also well established in some embodiments of the present invention.

Further, in some other embodiments of the present invention DU145 cell line revealed dose-dependent elevation of [Ca²⁺]c upon D-allose treatments. Studies from other laboratories have shown that poor control of Ca²⁺ homeostasis leads to moderate rise in [Ca²⁺]c, result in apoptotic cell death. In human prostate cancer cell line, the release of [Ca²⁺]c regulates cyt C secretion, which further plays a role in post-mitochondrial events involved in downstream caspase 3 activation leading to induction of programmed cell death. On the other hand the anti-apoptotic protein Bcl-2 prevents the leakage of Ca²⁺ from its store in endoplasmic reticulum (ER), thereby regulates [Ca²⁺]c. Therefore, experimental data as mentioned above, indicating the execution of apoptotic cell death in human HRPC cell line, DU145 through mitochondria mediated cyt C-caspase 3-PARP pathway is triggered by an increase in [Ca²⁺]c.

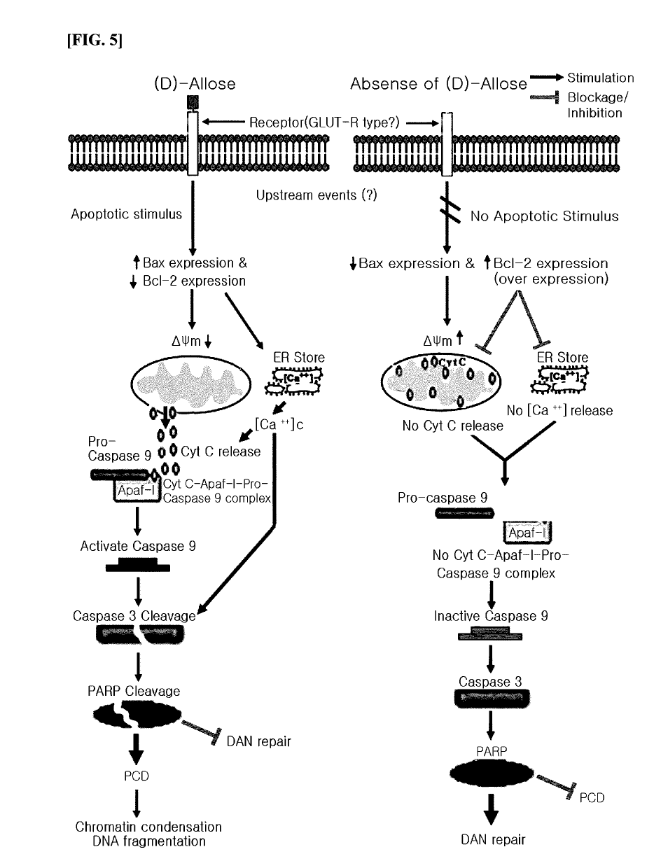

To understand the antiproliferative effect of D-allose through induction of programmed cell death in human HRPC cell line, DU145, one aspect of the present invention is schematically represented in FIG. 5. Therefore, results of in some embodiments of the present invention showed that D-allose inhibits DU145 cell growth by induction of programmed cell death through the Bcl-2 family proteins, Bax and Bcl-2, and the mitochondria mediated intrinsic apoptotic pathway members like cyt C, caspase 3 and PARP.

The elevation of [Ca²⁺]c plays a crucial role during early commitment of this intrinsic apoptotic pathway. Further, D-allose revealed no remarkable cytotoxic effect on human NPE cell line, PrEC. Therefore, the present inventors finally suggest the role of D-allose as a promising antiproliferative agent in late stage PC, may be without any side effects on normal prostate cells. This is the first estimate that D-allose induces programmed cell death in human HRPC cell lines in vitro. Further mechanism-based in vivo studies on anticancer activities with D-allose in animal models as well as in prostate biopsy samples are warranted.

Hereinafter, the preferred Examples are provided for better understanding. However, these Examples are for illustrative purpose only, and the invention is not intended to be limited by these Examples.

### EXAMPLE 1

### Cell Culture and Treatments

DU145 (human HRPC cell line, derived from a patient with brain metastasis) and PrEC (human normal prostate epithelial cell line) cell lines were obtained from Korea Cell Line Bank (KCLB, Seoul, South Korea). DU145 and PrEC cell lines were grown in triplicate culture plates containing RPMI-1640 (HyClone, Logan, Utah, USA) and Dulbecco's Modified Eagle's Medium (HyClone, Logan, Utah, USA) respectively, and kept in a humidified atmosphere containing 5% CO2 at 37° C. for 72 h. The media were supplemented with 10% heat-inactivated fetal bovine serum (HyClone, Logan, Utah, USA), 1% antibio-antimicotic solution containing 10,000 units/ml of penicillin and 10 mg/ml of streptomycin (HyClone, Logan, Utah, USA), HEPES, sodium bicarbonate, p-aminobenzoic acid and insulin (complete culture media). D-Allose was purchased from Sigma (St. Louis, Mo., USA) and dissolved at a concentration of 1 M in sterile phosphate-buffered saline (PBS), and was further sterilized by filtration. To examine the dose- and time-dependent effects, the cell lines (70-80% confluent) were treated with 0-40 mM of D-allose for up to 72 h, whereas without D-allose treated cell lines (0 mM) were considered as control. DU145 cell line was also treated with 0-40 mM of D-glucose. All the other chemicals used in the work were procured from Sigma (St. Louis, Mo., USA), unless otherwise specified.

### EXAMPLE 2

### Cell Viability

The logarithmic growth phase of DU145 and PrEC cell lines were taken for growth assay using 3-[4,5-dimethylthiazol-2-yl]-2,5-diphenyl tetrazolium bromide (MTT). The cell lines were seeded onto 96-well plates with 1×10⁵ cells/well in 200 μl of complete culture media containing 0, 1, 5, 10, 20 and 40 mM of D-allose and D-glucose for 24, 48 and 72 h. After incubation for the specified time at 37° C. in a humidified 5% CO2 incubator, cell viability was determined. MTT (5 mg/ml in PBS) was added to each well and incubated for additional 4 h at 37° C. To achieve solubilization of formazan crystal formed in viable cells, DMSO was added to each well, and the absorbance was recorded on a microplate reader at 595 nm wavelength. The effect of sugars on growth inhibition was assessed as percent cell viability where control cells were taken as 100% viable.

### EXAMPLE 3

### Detection and Quantification of Apoptosis

The Annexin-V-FLUOS Staining Kit (Roche Diagnostics GmbH, Mannheim, Germany) was used for the detection and quantification of the apoptotic cells. This kit uses a dual-staining protocol in which the apoptotic cells are stained with annexin-V (green fluorescence) and the necrotic cells are stained with propidium iodide (PI, red fluorescence). Briefly, DU145 cell line was grown at a density of 1×10⁶ cells in triplicate culture plates and then treated with D-allose (0, 20 and 40 mM) for 48 and 72 h. The cells were treated with trypsin and harvested with PBS and processed for labeling with annexin-V and PI by the use of the kit according to the manufacturer's protocol. The fluorescence was measured by flow cytometric analysis, performed on a FACSCalibur (Becton Dickinson, San Jose, Calif., USA) using 488 nm excitation and a 515 nm band pass filter for fluorescein detection and a filter [600 nm for PI detection (FL1: Annexin-V-FLUOS; FL2: PI). Further, apoptotic indices were measured by analysis of fragmented or scattered nuclei and condensed chromosome as the stringent morphological criteria of apoptosis, detectable by confocal microscopy on cells stained with DNA-intercalating dye 4-6-diamino-2-phenylindole (DAPI). For this purpose, DU145 and PrEC cell lines were exposed to 0, 20 and 40 mM of D-allose for up to 72 h, stained with DAPI (1 μg/ml in methanol), mounted with Prolong Antifade reagent (Molecular Probes, Eugene, Oreg., USA), and images were acquired by use of a confocal laser scanning microscope (Fluoview FV 1000, Olympus, Japan). The numbers of apoptotic nuclei per 100 cells were counted from the five high power fields/slide and apoptotic indices were expressed as a percentage of the untreated controls in each cell line, considering the value as 100%. DU145 cell line was also treated with 0-40 mM of D-glucose for determination of apoptotic indices as mentioned above.

### EXAMPLE 4

### Cell Cycle Analysis

DU145 cell line (1×10⁶ cells in triplicate culture plates) was treated with D-allose (0, 20 and 40 mM) for 48 and 72 h. The cells were treated with trypsin and harvested with PBS, centrifuged and resuspended the pellet in cold PBS-methanol mixture (1:9) for 1 h at 4° C. The cells were centrifuged, pellet washed and resuspended with PBS again, and incubated with RNAse (20 mg/ml in PBS) for 30 min at 37° C. The cells were chilled over ice and stained with PI (1 mg/ml in PBS) for 1 h and analyzed by flow cytometry using CellQuest software, version 3.0, performed on a FACSCalibur, fluorescence (FL2-A) detector equipped with 488 nm argon laser light source and 623 nm band pass filter (Becton Dickinson, San Jose, Calif., USA). Total 10,000 cells were acquired for analysis by CellQuest software and the quantification of FACS data was performed using ModFit LT software, version 2.0, (Becton Dickinson, San Jose, Calif., USA). The cell population was gated according to the characteristics side scatter and forward scatter intensities of FACS. The number of all gated living cells was taken as 100%, from which the percentage of cells in G0-G1, S and G2-M phase was calculated and presented in the right side of the histogram.

### EXAMPLE 5

### Protein Isolation and Western Blot Analysis

After 0, 20 and 40 mM of D-allose treatments for 72 h, DU145 cell line (1×10⁶ cells in triplicate culture plates) was harvested into cell lysis buffer (Cell Signaling, Danvers, Mass., USA) supplemented with 1 mM PMSF. The cells were then sonicated, centrifuged and protein concentration was measured by Bradford assay with the Bio-Rad protein assay solution using bovine serum albumin (BSA) as standard. Proteins (30 μg) were separated on a 12.5% SDS-PAGE under reducing conditions and transferred onto a PVDF membrane (Santa Cruz Biotechnology, Santa Cruz, Calif., USA). Prestained protein marker, broad range: 6-175 kDa (New England Biolabs Inc., Ipswich, Mass., USA) run in parallel for detection of the molecular weights of the proteins. Membrane was blocked with 5% (w/v) skimmed milk in order to reduce non-specific binding and immunoblotting was performed using rabbit derived anti-Bcl-2, anti-Bax, anti-Caspase 3 and anti-PARP antibodies (1:500; Santa Cruz Biotechnology, Santa Cruz, Calif., USA). Anti-β-actin antibody (1:500; Sigma, St. Louis, Mo., USA) was taken as control to confirm uniform loading. Membrane was probed with a goat derived horseradish peroxidase-conjugated anti-rabbit IgG (1:1000; Santa Cruz Biotechnology, Santa Cruz, Calif., USA), and proteins were detected by the enhanced chemiluminescence (ECL) detection system according to the vendor's protocol (Amersham Biosciences, Piscataway, N.J., USA) followed by X-ray exposure. The densitometry analysis of the protein bands were performed by Sigma Gel, version 1.0 (Jandel Scientific, San Rafael, Calif., USA).

### EXAMPLE 6

### Calculation of Bcl-2/Bax Ratio

Bcl-2/bax ratios of D-allose treated groups were calculated on the basis of the densitometric analysis of the corresponding protein bands and compared the values with that of the untreated control.

### EXAMPLE 7

### RNA Isolation and Reverse Transcription-Polymerase Chain Reaction (RT-PCR)

To identify the relative levels of expression of Bcl-2, Bax and Caspase 3 mRNAs after 0, 20 and 40 mM of D-allose treatments for 72 h, RT-PCR was performed. DU145 cell line (1×10⁶ cells in triplicate culture plates) was harvested as mentioned above and total RNA was extracted using TRIzol reagent (Invitrogen, Carlsbad, Calif., USA) following a standard laboratory protocol. The RNA purity and concentrations were determined. Then 2 μg of total RNA was reversely transcribed to single-stranded cDNA using Oligo(dT)12-18 primer (Invitrogen, Carlsbad, Calif., USA) with M-MLV reverse transcriptase (Promega, Madison, Wis., USA). For PCR reaction, 4 μl of cDNA was reacted with 20 pmol each of the forward and reverse primers (Bioneer Corporation, Seoul, South Korea), and GoTaq® Green Master Mix 29 containing PCR buffer, 25 mM magnesium chloride, 10 mM dNTP mix and Taq enzyme (Promega, Madison, Wis., USA), and amplified. The human primers of each transcript were as follows: Bcl-2 [5′-CGACGACTTC TCCCGCCGCTACCGC-3′ (forward) (SEQ ID NO: 1) and 5′-CCGCATGC TGGGGCCGTACAGTTCC-3′ (reverse) (SEQ ID NO: 2)], Bax [5′-GTGCACCAAGGTGCCGGAAC-3′ (forward) (SEQ ID NO: 3) and 5′-TCAGCCCA TCTTCTTCCAGA-3′ (reverse) (SEQ ID NO: 4)], Caspase 3 [5′-AGTGCTCGCAGCTCATACCT-3′ (forward) (SEQ ID NO: 5) and 5′-GAGTCCATTGATTCGCTTCC-3′ (reverse) (SEQ ID NO: 6)], and β-actin (as loading control) [5′-GTGGGGCGCCCCAGGCACCA-3′ (forward) (SEQ ID NO: 7) and 5′-CTCCTTAATGTCACGCACGATTTC-3′ (reverse) (SEQ ID NO: 8)]. The conditions for PCR were as followings: initial denaturation at 94° C. for 5 min; 25 cycles of denaturation at 94° C. for 1 min, annealing for 1 min (Bcl-2: 68° C., Bax: 53° C., Caspase 3: 55° C., and β-actin: 63° C.) and elongation at 72° C. for 1 min; and another final extension step at 72° C. for 10 min on a PC-812 Thermal Cycler (Astec, Fukuoka, Japan). The PCR products were electrophoresed in 1% agarose gel containing ethidium bromide (1 μl/ml in PBS) for 25 min and exposed to UV lamp for photography of the bands. The molecular sizes of the amplified products were determined by comparison with the molecular weight marker (100 by DNAladder; Promega, Madison, Wis., USA) run parallel with the PCR products. The densities of mRNA bands were analyzed by Molecular Analyst™, version 1.4.1 (Bio-Rad, Hercules, Calif., USA).

### EXAMPLE 8

### Visualization of Mitochondrial Cyt C Release and Nuclear Morphology

The in situ analysis of cyt C was carried out by immunofluorescence technique. Briefly, DU145 cell line (1×10⁶ cells in triplicate culture plates) was treated with D-allose (0, 20 and 40 mM for 48 and 72 h) and was fixed with 4% paraformaldehyde, permeabilized with 0.1% Triton and washed with PBS in chilled condition. Cyt C was detected by using mouse anti-cyt C antibody and rabbit anti-mouse FITC-labeled antibody (1:250 and 1:200, respectively; Santa Cruz Biotechnology, Santa Cruz, Calif., USA). DU145 cell line treated with FITC-labeled secondary antibody alone was considered as negative control. Subsequently, chromatin was stained with PI (1 mg/ml in PBS) for 20 min in dark and slides were mounted with Prolong Antifade reagent (Molecular Probes, Eugene, Oreg., USA). Cyt C (green) and chromatin (red) staining patterns were acquired by use of a confocal laser scanning microscope (Fluoview FV 1000, Olympus, Japan).

### EXAMPLE 9

### Measurement of Mitochondrial Δψₘ

Mitochondrial Δψm was measured by JC-1 mitochondrial membrane potential detection kit (Biotium Inc, Hayward, Calif., USA) according to the manufacturer's protocol. Briefly, DU145 cell line (1×10⁶ cells in triplicate culture plates) was harvested after treatments with 0, 20 and 40 mM of D-allose for up to 72 h as mentioned above, stained with 19 JC-1 reagent at 37° C. for 15 min and resuspended twice in 1× assay buffer. Changes in mitochondrial Δψm were measured at the single cell level by FACSCalibur flow cytometer (Becton Dickinson, San Jose, Calif., USA) under the following conditions: FL1, 511 volts; FL2, 389 volts; FL1-10.5% FL2; FL2-25.9% FL1; 488 nm argon excitation laser and 585 nm band pass filter. In total 10,000 cells were acquired for analysis by CellQuest software, version 3.0 (Becton Dickinson, San Jose, Calif., USA), and the quantification of cells with low mitochondrial Δψm as the percentage of total cell population was performed. Further, the DU145 cell line was seeded onto chamber slides with 1×10⁵ cells/chamber in 500 μl of complete culture medium containing 0, 20 and 40 mM of D-allose for 72 h, permeabilized with 0.1% Triton, stained with 1×JC-1 reagent in dark, mounted with Prolong Antifade reagent (Molecular Probes, Eugene, Oreg., USA), and images (JC-1, green) were acquired by use of a fluorescence microscope (Axioplan 2, Carl Zeiss, Germany).

### EXAMPLE 10

### Fura-2 Measurement of [Ca²⁺]_{c}

[Ca²⁺]c was measured by fluorescent indicator fura-2 acetoxymethyl ester (fura-2 AM) as described in Malgaroli A et al., J. Cell. Biol. 105:2145-2155, 1987. Briefly, DU145 cell line (1×10⁶ cells in triplicate culture plates) was exposed to 0, 20 and 40 mM of D-allose for 48 and 72 h and then incubated with serum-free culture media containing 5 μM of fura-2AM at 37° C. in dark for another 1 h. The cells were washed twice with Locke's solution (pH 7.3) and the fura-2 fluorescence signals of [Ca²⁺]c were measured by luminescence spectrophotometer (LS 50B, Perkin Elmer, Boston, Mass., USA) using 340 and 380 nm as excitation wavelengths (λex), and 510 nm as emission wavelength (λem). The [Ca²⁺]c concentration was derived from the ratio of the fluorescence intensities for each of the excitation wavelengths as mentioned above, and from the Grynkiewicz equation:

$${[ {Ca}^{2 +} ]}_{c} = {K}_{d} \times \frac{( R - {R}_{min} )}{{R}_{max} - R} \times \frac{{S}_{f 2}}{{S}_{b 2}}$$

where Kd: Dissociation constant of the fura-2-Ca²⁺ interaction to be 225 nM in the intracellular environment; R: Fluorescence ratio at 340 and 380 nm; Rmin: Ratio with zero Ca²⁺; Rmax: Ratio with saturating Ca²⁺ (using calcium chloride); Sf2: Fluorescence at 380 nm with zero Ca²⁺; and Sb2: Fluorescence at 380 nm with saturating Ca²⁺. Further, DU145 cell line was seeded onto chamber slides with 1×10⁵ cells/chamber in 500 μl of serum-free culture media containing 5 μM of fura-2 AM for fura-2 images of [Ca²⁺]c, with and without D-allose treatments (0 and 20 mM) for 48 h. The slides were mounted with Prolong Antifade reagent (Molecular Probes, Eugene, Oreg., USA), and images (JC-1, green) were acquired by use of a fluorescence microscope (Axioplan 2, Carl Zeiss, Germany).

It is an effect of some embodiments of the present invention to provide the dose and time-dependent effects of D-allose on the proliferation of late stage human HRPC using DU145 cell line in vitro, which are highly tumorigenic and chemotherapy-resistant. Also, it is an effect of some other embodiments of the present invention to provide the mechanism of D-allose induced apoptosis in DU145 cell line by modulation of pro- and anti-apoptotic Bcl-2 family members, Bax and Bcl-2, in favor of execution of programmed cell death, which is accompanied by mitochondrial cyt C release along with concomitant alteration of mitochondrial Δψm, elevation of [Ca²⁺]c, and cleavage of caspase-3 and poly (ADP-ribose) polymerase (PARP).

What is claimed is:

1. A composition for inducing apoptosis in prostate cancer cells, the composition comprising D-allose.

2. The composition according to claim 1, wherein said prostate cancer cells comprise human hormone refractory prostate cancer cells.

3. The composition according to claim 1, wherein said D-allose induces apoptosis in prostate cancer cells by i) lowering a mitochondrial transmembrane potential (Δψₘ); ii) elevating a calcium concentration in cytosol ([Ca²⁺]_{c}); iii) cleaving caspase 3 and poly (ADP-ribose) polymerase (PARP); and iv) releasing cytochrome C (cyt C).

4. The composition according to claim 1, wherein said D-allose induces apoptosis by modulating expression at a transcription level of apoptosis-related genes through increasing expression of Bax and decreasing expression of Bcl-2.

5. A method of inducing apoptosis in prostate cancer cells, the method comprising contacting the composition of claim 1 to prostate cancer cells.

6. The method according to claim 5, wherein said composition is applied at 20˜40 mM per 1×10⁵ cells of prostate cancer cells.

7. A pharmaceutical composition for the prevention or treatment of prostate cancer by inducing apoptosis and inhibiting cell growth of prostate cancer cells, the composition comprising D-allose and a pharmaceutically acceptable carrier.

8. The pharmaceutical composition according to claim 7, wherein said prostate cancer is hormone refractory prostate cancer.

9. A composition for health functional food by inducing apoptosis in prostate cancer cells to prevent and improve prostate cancer, the composition comprising food, D-allose and a sitologically acceptable food additive.

10. A method of treating prostate cancer, the method comprising administering, to a person in need of such treatment, D-allose in an amount sufficient to induce apoptosis and inhibiting cell growth of prostate cancer cells.

11. The method according to claim 10, wherein said prostate cancer is hormone refractory prostate cancer.
